(19) United States
(12) Patent Application Publication
(10) Pub. No.: US 2025/0051711 A1
(43) Pub. Date: Feb. 13, 2025

(54) CELL CULTURE DEVICE FOR PROMOTING EXOSOME SECRETION

(71) Applicant: Cellabsys Inc., Incheon (KR)

(72) Inventor: Minah Yang, Incheon (KR)

(73) Assignee: Cellabsys Inc., Incheon (KR)

(21) Appl. No.: 18/384,891

(22) Filed: Oct. 30, 2023

(30) Foreign Application Priority Data
Aug. 7, 2023 (KR) 10-2023-0102855

(51) Int. Cl. C12M 1/42; C12M 1/00; C12M 1/12; C12M 1/34

(52) U.S. Cl. CPC C12M 35/04; C12M 23/04; C12M 29/08; C12M 41/40

(57) ABSTRACT

The present disclosure provides a cell culture device which includes a case having a space for culturing cells, a culture case placed inside the case and storing a culture solution including cells to be cultured, a fixing portion placed inside the case and configured to pressurize and fix an upper surface and a lower surface of the culture case, and an ultrasonic generator placed on at least one surface of the fixing portion and applying vibration generated by ultrasonic waves to the culture case and which promotes exosome secretion from cells by applying vibration generated by ultrasonic waves to cells to be cultured and improves the degree of ultrasonic vibration transfer by applying ultrasonic waves while fixing upper and lower surfaces of a culture case where cells are cultured.

### CROSS-REFERENCE TO RELATED APPLICATION

This application is based on and claims priority under 35 U.S.C. § 119 to Korean Patent Application No. 10-2023-0102855 filed in August 7, 2023, in the Korean Intellectual Property Office, the disclosure of which is incorporated by reference herein in its entirety.

### BACKGROUND

The present disclosure relates to a cell culture device for promoting exosome secretion, and more particularly, to a cell culture device for promoting exosome secretion that improves exosome secretion by applying physical stimulation to stem cells.

Exosomes are nano-vesicles secreted by cells for information exchange between cells and contain various bioactive substances.

In particular, stem cell-derived exosomes have ability to differentiate and regenerate through various proteins and miRNAs, and are known to be widely applicable to regeneration and treatment of various cells and tissues.

Exosomes are small vesicles having a membrane structure secreted by various types of cells. A diameter of exosome is reported to be approximately 30 nm to 150 nm. In a study using an electron microscope, it was observed that exosome does not drop directly out of the plasma membrane, but originate from a specific section within a cell called multivesicular bodies (MVBs) and are released and secreted outside the cell. That is, when fusion of the multivesicular body and the plasma membrane occurs, the vesicles are released to the environment outside cells, which are called exosomes.

Exosomes contain various components, such as receptors, proteins, and miRNA, and accordingly, the exosomes are known to play an important role in signaling between cells. Also, exosomes contain relatively less animal serum than stem cells, and accordingly, the risk of zoonosis caused by animal serum infection may also be removed. Considering the characteristics of exosomes, treatment using the exosomes is expected to become a new paradigm that may overcome limitations of existing stem cell treatments.

Therefore, there is a need for research on technology to promote exosome secretion by applying physical stimulation to stem cells as a method of obtaining exosomes in large quantities.

The related art for the present disclosure may include Patent Registration No. 10-1798726.

### SUMMARY

The present disclosure provides a cell culture device for promoting exosome secretion which promotes exosome secretion from cells by applying vibration generated by ultrasonic waves to cells to be cultured.

According to an aspect of the present disclosure, a cell culture device includes a case having a space for culturing cells, a culture case placed inside the case and storing a culture solution including cells to be cultured, a fixing portion placed inside the case and configured to pressurize and fix an upper surface and a lower surface of the culture case, and an ultrasonic generator placed on at least one surface of the fixing portion and applying vibration generated by ultrasonic waves to the culture case.

The case may include a chamber having a bottom surface and a wall surface surrounding the bottom surface and including the fixing portion, the ultrasonic generator, and the culture case, and a cover detachably coupled to at least one surface of the chamber.

The chamber may include a separation portion configured to separate a culture space where the culture case and the fixing portion are placed from a control space other than the culture space, and the culture space may be sealed by the separation portion and the cover.

The fixing portion may include a support portion configured to support a lower surface of the culture case, and a pressing portion configured to support an upper surface of the culture case, and the pressing portion may pressurize the upper surface of the culture case as the cover is closed.

The supporting portion may include the support portion is formed to separate the culture case from the bottom surface of the chamber.

The supporting portion may include a plate that is in contact with the culture case and has an upper surface on which a placement groove corresponding to the culture case is formed, an elastic body configured to support the plate from the bottom surface of the chamber; and an absorption pad placed between the elastic body and the bottom surface of the chamber.

The culture case may include a plurality of unit culture cases which have the same shape and of which side areas gradually increase, the placement groove may include a plurality of placement grooves respectively corresponding to the side areas of the plurality of unit culture cases, and one of the plurality of placement grooves may be placed inside the placement groove of the culture case having a larger side area.

The ultrasonic generator may include a first ultrasonic vibrator placed on a lower surface of the plate and configured to generate ultrasonic waves, and a power supply configured to supply power to the ultrasonic vibrator.

The pressing portion may include a pressing plate configured to pressurize the upper surface of the culture case, a connector connected to the cover, an elastic body connecting the pressing plate to the connector, and an absorption pad placed between the elastic body and the connector.

The ultrasonic generator may further include a second ultrasonic vibrator placed on an upper surface of the pressing plate and configured to generate the ultrasonic waves by receiving the power from the power supply.

The first and second ultrasonic vibrators may each include a plurality of ultrasonic vibrators configured in a matrix structure.

### BRIEF DESCRIPTION OF THE DRAWINGS

Embodiments will be more clearly understood from the following detailed description taken in conjunction with the accompanying drawings in which:

FIG. 1 is a cross-sectional view illustrating a configuration of a cell culture device for promoting exosome secretion, according to an embodiment of the present disclosure;

FIG. 2 is a perspective view illustrating a configuration of a rear surface of the cell culture device for promoting exosome secretion illustrated in FIG. 1;

FIG. 3 is a view illustrating an open state of a cover of the cell culture device for promoting exosome secretion illustrated in FIG. 1;

FIG. 4 is a view illustrating an example of a configuration of an opening/closing rail illustrated in the cell culture device for promoting exosome secretion illustrated in FIG. 1;

FIG. 5 is a perspective view illustrating a configuration of a placement groove formed in a plate used in the present disclosure;

FIG. 6 is a cross-sectional view taken along line A-A in FIG. 5;

FIG. 7 is a bottom perspective view illustrating an example of a configuration of a plate used in the present disclosure;

FIG. 8 is a view illustrating an example of a state in which a first unit culture case is placed in the first unit placement groove illustrated in FIG. 6;

FIG. 9 is a view illustrating a state in which a fourth unit culture case is placed in a fourth unit placement groove; and

FIG. 10 is a view illustrating pressurization by a pressing plate after the fourth unit culture case is placed in the fourth unit placement groove.

### DETAILED DESCRIPTION OF THE EMBODIMENTS

Hereinafter, preferred embodiments according to the present disclosure will be described in detail with reference to the attached drawings.

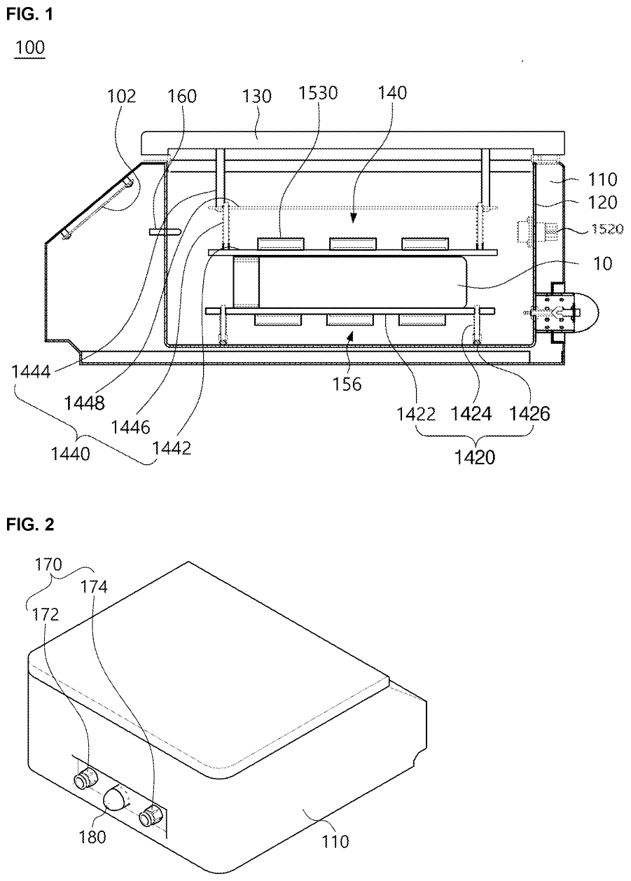

FIG. 1 is a cross-sectional view illustrating a configuration of a cell culture device for promoting exosome secretion, according to an embodiment of the present disclosure.

Referring to FIG. 1, a cell culture device 100 for promoting exosome secretion, according to an embodiment of the present disclosure includes a case 110, a culture case 10, a chamber 120, a cover 130, a fixture 140, and an ultrasonic generator 150.

The case 110 has a rectangular parallelepiped shape with a preset size.

A preset space is provided inside the case 110 such that the culture case 10 to be described below may be placed.

The culture case 10 has a rectangular parallelepiped shape with a preset size, and a preset amount of a culture solution is stored inside the culture case 10.

The culture case 10 may have one end that includes a flask including a culture solution doorway through which the culture solution may enter and exit and a stopper placed at the culture solution doorway.

The culture case 10 may be divided into T25, T75, T150, and T225 according to the size of a side area. A culture case used in the present disclosure may include a plurality of unit culture cases with different sizes of side areas. Here, the culture case 10 and T25, T75, T150, and T225 indicating a size of the culture case 10 correspond to the known technology, and thus, detailed description thereof is omitted.

A control panel 102 may be placed on the front of the case 110 to control an operation of the cell culture device 100 for promoting exosome secretion according to the present disclosure. Here, in order to easily determine a display item displayed on the control panel 102, the front of the case 110 may be inclined at a preset inclination.

A pressure adjustment unit 170, which is described below, may be placed on the back of the case 110.

Here, the culture case 10 may be placed inside the case 110, and the chamber 120 and the cover 130, which are described below, may be placed to be isolated from an external space.

The chamber 120 includes a bottom surface and a wall surrounding the bottom surface, has a preset size, and has a rectangular parallelepiped shape with an open upper portion. The culture case 10, the fixture 140, and the ultrasonic generator 150, which are described below, may be placed inside the chamber 120.

The chamber 120 serves as a separation unit that divides the inside of the case 110 into a culture space where the culture case 10 and the fixture 140 are placed and a control space other than the culture space.

The cover 130 has a flat plate shape with a preset area and thickness, and is detachably placed in an open region of an upper portion of the chamber 120.

When the cover 130 is closed, the inside of the chamber 120 is isolated from the outside.

Here, one side of the cover 130 may be opened and closed by rotating around one side of the upper portion of the chamber 120.

FIG. 4 is a view illustrating a configuration of a cover used in the present disclosure.

The cover 130 further includes an opening/closing rail 132.

One end of the opening/closing rail 132 is connected to the one side of the upper portion of the chamber 120, the other end is connected to one side of a lower portion of the cover 130, and the middle is bent at a preset angle. In addition, one end of the opening/closing rail 132 connected to one side of the upper portion of the chamber 120 is bent, and a straight portion 132a extending to a middle portion with a preset length is formed.

When the cover 130 is lifted from the upper portion of the chamber 120 while the chamber 120 is sealed by the cover 130, the opening/closing rail 132 rotates after the cover 130 ascends while moving in a straight line a distance corresponding to a straight length of a straight portion 132a.

When the cover 130 is separated from the chamber 120, a pressing plate 1442, which is described below, may be separated from an upper surface of the culture case 10. In addition, when the cover 130 is coupled to the upper portion of the chamber 120 and a culture space inside the chamber 120 is isolated from the outside, the pressing plate 1442 may apply a preset pressure to an upper surface of the culture case 10 to closely adhere to the upper surface.

The fixing portion 140 is placed inside the chamber 120 and pressurizes and fixes an upper surface and a lower surface of the culture case 10.

The fixing portion 140 includes a support portion 1420 and a pressing portion 1440.

The support portion 1420 supports the culture case 10 placed inside the chamber 120 from a lower portion.

The support portion 1420 includes a plate 1422, a first elastic body 1424, and a first absorption pad 1426.

The plate 1422 may be formed in the form of a flat plate with a preset area. The plate 1422 is placed at a position separated a preset distance from a bottom surface inside the chamber 120.

An upper surface of the plate 1422 is in contact with a lower surface of the culture case 10, which is described below to support the lower surface.

Here, a placement groove 1423 corresponding to a size of the culture case 10 may be formed at a preset depth on an upper surface of the plate 1422.

When the cell culture device 100 for promoting exosome secretion according to the present disclosure is in operation, that is, when a preset vibration is applied to the culture case 10, the culture case 10 placed in the placement groove 1423 may maintain a state of being placed in the placement groove 1423.

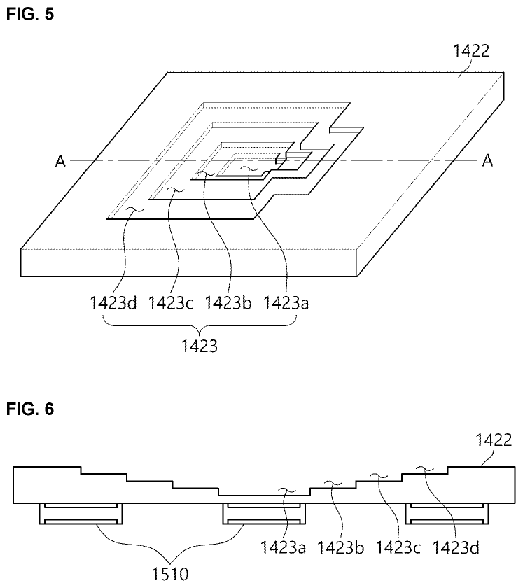

FIG. 5 is a perspective view illustrating a configuration of a placement groove formed in a plate used in the present disclosure, and FIG. 6 is a cross-sectional view taken along line A-A in FIG. 5.

Here, the unit culture cases respectively corresponding to T25, T75, T150, and T225 will be referred to as first, second, third, and fourth unit culture cases 10a, 10b, 10c, and 10d.

The placement groove 1423 on an upper surface of the plate 1422 may include first, second, third, and fourth unit placement grooves 1423a, 1423b, 1423c, and 1423d respectively corresponding to the first, second, third, and fourth unit culture cases 10a, 10b, 10c, and 10d.

In the drawings, the first, second, third, and fourth unit placement grooves 1423a, 1423b, 1423c, and 1423d may be formed as follows.

The fourth unit placement groove 1423d corresponding to the fourth unit culture case 10d is formed at a preset depth in the center of an upper surface of the plate 1422, and the third unit placement groove 1423c corresponding to the third unit culture case 10c may be formed at a preset depth in the center of the fourth unit placement groove 1423d. In addition, the second unit placement groove 1423b corresponding to the second unit culture case 10b may be formed at a preset depth in the center of the third unit placement groove 1423c, and the first unit placement groove 1423a corresponding to the first unit culture case 10a may be formed at a preset depth in the center of the second unit placement groove 1423b.

As described above, the first, second, third, and fourth unit placement grooves 1423a, 1423b, 1423c, and 1423d may each be formed in the form of a concentric circle sharing a central point.

In this case, it can be seen that a formation depth gradually increases from the fourth unit placement groove 1423d to the first unit placement groove 1423a.

The drawings illustrate that a difference in depth between the unit placement grooves is formed to correspond to ⅙ of a thickness of the plate 1422, but this is for an easy-to-understand expression of the difference in depth between the unit placement grooves, and in reality, it is preferable that a culture case is small enough to easily check a placement position when the culture case is placed in a unit placement groove.

Also, the first, second, third, and fourth unit placement grooves 1423a, 1423b, 1423c, and 1423d may be formed in different ways depending on a user's needs. For example, the first, second, third, and fourth unit placement grooves 1423a, 1423b, 1423c, and 1423d may be formed adjacent to each other on an upper surface of the plate 1422.

The first elastic body 1424 is formed in the form of a rod having a preset length, one end of the first elastic body 1424 is connected to a bottom surface of the chamber 120, and the other end of the first elastic body 1424 is connected to an edge of the plate 1422.

The first elastic body 1424 prevents vibration caused by ultrasonic waves generated by the ultrasonic generator 150 from being applied to the chamber 120.

Due to this, it is preferable that the first elastic body 1424 includes a non-vibration spring.

The first absorption pad 1426 is placed at the bottom of the first elastic body 1424 and absorbs vibration that exceeds a vibration absorption degree of the non-vibration spring.

The pressing portion 1440 pressurizes the culture case 10 placed inside the chamber 120 from the top.

The pressing portion 1440 includes a pressing plate 1442, a connector 1444, a second elastic body 1446, and a second absorption pad 1448.

The pressing plate 1442 may be formed in the form of a plate with a preset area. The pressing plate 1442 may be placed on an upper portion of the culture case 10 to pressurize the culture case 10.

The connector 1444 is formed in the form of a rod having a preset length. A plurality of connectors 1444 are placed at regular intervals on a lower surface of the cover 130, and the plurality of connectors 1444 connect the cover 130 to the pressing plate 1442, and thereby, the pressing plate 1442 pressurizes the culture case 10 according to opening and closing of the cover 130.

The second elastic body 1446 is made to have a preset length, one end of the second elastic body 1446 is connected to the connector 1444, and the other end of the second elastic body 1446 is connected to the pressing plate 1442, and accordingly, vibration generated by the ultrasonic generator 150, which is described below, is prevented from being transferred to the cover 130 side.

The second absorption pad 1448 is placed between the connector 1444 and the second elastic body 1446 to absorb the vibration applied from the outside.

The ultrasonic generator 150 generates ultrasonic waves and applies the ultrasonic waves to the culture case 10 to promote exosome secretion in stem cells being cultured.

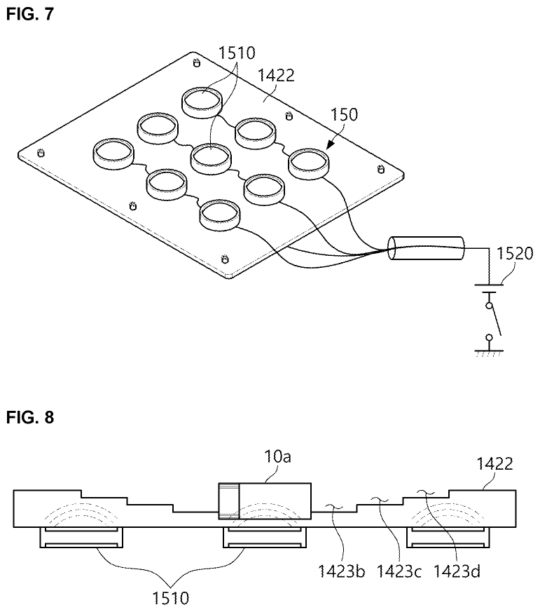

FIG. 7 is a bottom perspective view illustrating an example of a configuration of a plate used in the present disclosure, and illustrates a configuration of the ultrasonic generator 150 placed on a lower surface of the plate 1422.

It can be seen that the ultrasonic generator 150 includes a first ultrasonic vibrator 1510 and a power supply 1520.

The first ultrasonic vibrator 1510 includes a plurality of unit ultrasonic vibrators placed on a lower surface of the plate 1422. Although FIG. 7 illustrates that the number of first unit ultrasonic vibrators is 9, but the number of first unit ultrasonic vibrators may be increased or decreased depending on a user's needs.

Here, the plurality of unit ultrasonic vibrators are arranged in a matrix.

The first ultrasonic vibrator 1510 receives external power and generates preset ultrasonic vibration, and the generated vibration is applied to the culture case 10 through the plate 1422.

The power supply 1520 supplies power necessary for ultrasonic generation in the first ultrasonic vibrator 1510.

Meanwhile, the ultrasonic generator 150 may further include a second ultrasonic vibrator 1530.

The second ultrasonic vibrator 1530 is placed on an upper surface of the pressing plate 1442 and generates ultrasonic vibration by using the power supplied by the power supply 1520, and the generated vibration is transferred to the culture case 10 through the pressing plate 1442.

The second ultrasonic vibrator 1530 may be configured in the same way as the first ultrasonic vibrator 1510, and accordingly, detailed description thereof is omitted.

Meanwhile, the present disclosure further includes a sensing unit 160, a pressure adjustment unit 170, and a controller 180.

The sensing unit 160 measures pressure, temperature, carbon dioxide concentration, and nitrogen concentration inside a culture space in the chamber 120 and outputs signals corresponding to the measured values.

The sensing unit 160 may include various measurement sensors, such as a temperature measurement sensor, a pressure measurement sensor, a carbon dioxide concentration measurement sensor, and a nitrogen concentration measurement sensor.

The sensing unit 160 may be placed to be partially inserted into the culture space through a wall of the chamber 120.

The pressure adjustment unit 170 adjusts the pressure in the chamber 120 by injecting gas into the culture space in the chamber 120 or discharging gas in the culture space to the outside of the chamber 120.

A component denoted by reference numeral 1520 in FIG. 1 is a connector included in the power supply to be described below.

Referring to FIG. 2, it can be seen that the pressure adjustment unit 170 includes a carbon dioxide input/output port 172 and a nitrogen input/output port 174 placed through a wall of the chamber 120.

When the controller 180 receives the signal output from the sensing unit 160 and determines that the measured value is different from a preset value, the controller 180 operates the pressure adjustment unit 170 to control the pressure inside the chamber 120.

The controller 180 operates the pressure adjustment unit 170 to maintain the pressure of a culture space in the chamber 120 at 1 psi to 2 psi. Here, the pressure of the culture space corresponds to a human blood pressure.

The controller 180 may be placed between the carbon dioxide input/output port 172 and the nitrogen input/output port 174.

The use of the cell culture device 100 for promoting exosome secretion according to the present disclosure will be described herein.

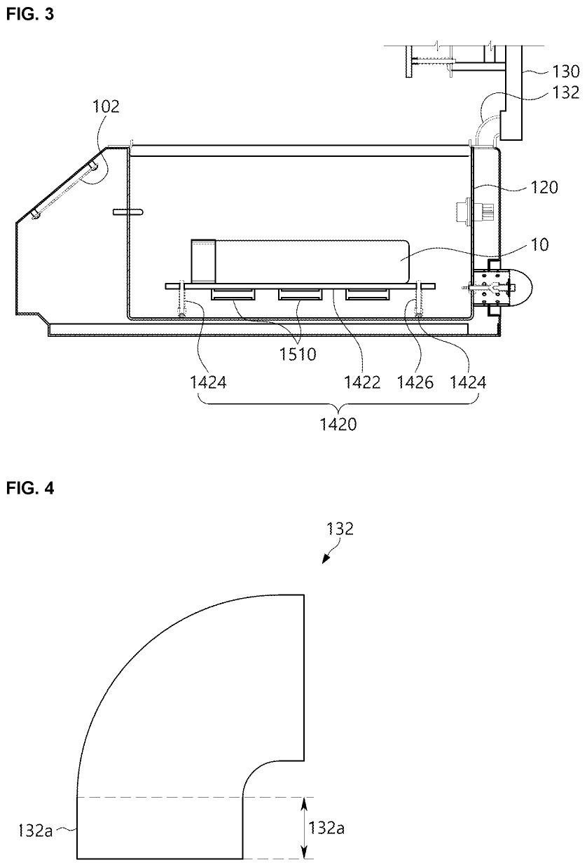

As illustrated in FIG. 3, after the cover 130 is opened, the first unit culture case 10a is placed in the first unit placement groove 1423a of the plate 1422 placed inside the chamber 120.

FIG. 8 illustrates a state in which the first unit culture case 10a is placed in the first unit placement groove 1423a.

Thereafter, the cover 130 is closed such that the pressing plate 1442 pressurizes an upper surface of the first unit culture case 10a.

Thereafter, the first ultrasonic vibrator 1510 operates to cause the vibration generated by ultrasonic waves to be applied to the first unit culture case 10a.

Here, the first ultrasonic vibrator 1510 generates ultrasonic waves of 30 kHz to 45 kHz for 10 seconds to 20 seconds.

When the above process is completed, a culture solution in the first unit culture case 10a is transferred to the second unit culture case 10b, and the second unit culture case 10b is placed in the second unit placement groove 1423b such that the ultrasonic vibration application process described above is performed.

Afterwards, the culture solution is sequentially transferred to the third unit culture case 10c and the fourth unit culture case 10d to perform the ultrasonic vibration application process.

Here, when placing the second unit culture case 10b, a preset distance may be between the second unit culture case 10b and the bottom of the first unit placement groove 1423a. This may be equally applied when arranging the third and fourth unit culture cases 10c and 10d.

The placement of the fourth unit culture case 10d will be described as an example.

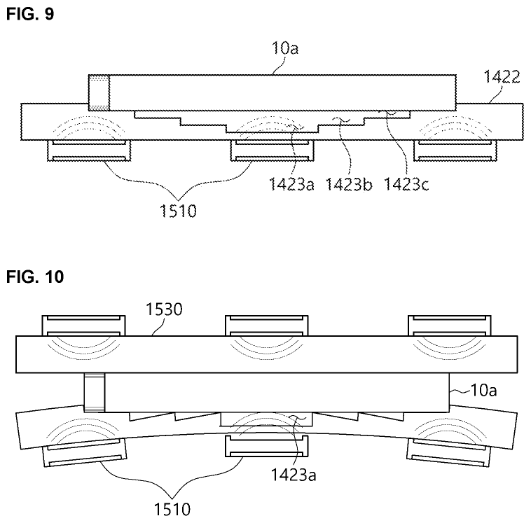

FIG. 9 is a view illustrating a state in which the fourth unit culture case 10d is placed in the fourth unit placement groove 1423d.

After the fourth unit culture case 10d is placed in the fourth unit placement groove 1423d, a user closes the cover 130.

Here, when the cover 130 is closed, as illustrated in FIG. 10, the pressing plate 1442 connected to the cover 130 pressurizes the fourth unit culture case 10d from the top, and an edge of the fourth unit culture case 10d pressurizes surroundings of the plate 1422 and deforms the plate 1422 to cause a lower surface of the fourth unit culture case 10d to come into contact with a boundary between the unit displacement grooves, and accordingly, vibration caused by ultrasonic waves generated by the first ultrasonic vibrator 1510 may be applied to the fourth unit culture case 10d.

Also, in an exosome production step using the fourth unit culture case 10d having the largest size among the culture cases, a culture solution is contained in the entire culture vessel, and accordingly, the first ultrasonic vibrator 1510 and the second ultrasonic vibrator 1530 operate together to apply ultrasonic vibration.

According to the present disclosure described above, vibration is generated by using ultrasonic waves, and the vibration physically stimulates culture cells, and thereby, exosome secretion of the culture cells may be promoted, and ultrasonic waves are applied to a culture case in which cells are cultured while upper and lower portions of the culture case are fixed, and thereby, the degree of transmission of ultrasonic vibration to a culture solution stored inside the culture case may be improved.

The present disclosure is described with reference to the embodiments illustrated in the drawings, but the embodiments are merely examples, and those skilled in the art to which the present disclosure belongs will understand that various modifications and equivalent other embodiments may be derived therefrom. Therefore, the true scope of technical protection of the present disclosure should be determined by the technical idea of following claims.

What is claimed is:

1. A cell culture device comprising:
a case having a space for culturing cells;
a culture case placed inside the case and storing a culture solution including cells to be cultured;
a fixing portion placed inside the case and configured to pressurize and fix an upper surface and a lower surface of the culture case; and
an ultrasonic generator placed on at least one surface of the fixing portion and applying vibration generated by ultrasonic waves to the culture case.

2. The cell culture device of claim 1, wherein the case includes:
a chamber having a bottom surface and a wall surface surrounding the bottom surface and including the fixing portion, the ultrasonic generator, and the culture case; and
a cover detachably coupled to at least one surface of the chamber.

3. The cell culture device of claim 2, wherein
the chamber includes a separation portion configured to separate a culture space where the culture case and the fixing portion are placed from a control space other than the culture space; and
the culture space is sealed by the separation portion and the cover.

4. The cell culture device of claim 3, wherein
the fixing portion includes a support portion configured to support a lower surface of the culture case, and a pressing portion configured to support an upper surface of the culture case, and
the pressing portion pressurizes the upper surface of the culture case as the cover is closed.

5. The cell culture device of claim 4, wherein
the support portion is formed to separate the culture case from the bottom surface of the chamber.

6. The cell culture device of claim 5, wherein the support portion includes:
a plate that is in contact with the culture case and has an upper surface on which a placement groove corresponding to the culture case is formed;
an elastic body configured to support the plate from the bottom surface of the chamber; and
an absorption pad placed between the elastic body and the bottom surface of the chamber.

7. The cell culture device of claim 6, wherein
the culture case includes a plurality of unit culture cases which have the same shape and of which side areas gradually increase,
the placement groove includes a plurality of placement grooves respectively corresponding to the side areas of the plurality of unit culture cases, and
one of the plurality of placement grooves is placed inside the placement groove of the culture case having a larger side area.

8. The cell culture device of claim 6, wherein the ultrasonic generator includes:
a first ultrasonic vibrator placed on a lower surface of the plate and configured to generate ultrasonic waves; and
a power supply configured to supply power to the ultrasonic vibrator.

9. The cell culture device of claim 8, wherein the pressing portion includes:
a pressing plate configured to pressurize the upper surface of the culture case;
a connector connected to the cover;
an elastic body connecting the pressing plate to the connector; and
an absorption pad placed between the elastic body and the connector.

10. The cell culture device of claim 9, wherein
the ultrasonic generator further includes a second ultrasonic vibrator placed on an upper surface of the pressing plate and configured to generate the ultrasonic waves by receiving the power from the power supply.

11. The cell culture device of claim 10, wherein
the first ultrasonic vibrator and the second ultrasonic vibrator each include a plurality of ultrasonic vibrators configured in a matrix structure.

12. The cell culture device of claim 10, wherein
the cover includes a rail portion configured to move a preset distance in a straight line when separated from the chamber, and
the pressing plate is separated from the culture case as the cover is separated from the chamber.

13. The cell culture device of claim 3, further comprising:
a sensing unit configured to measure pressure and temperature in the culture space; and
a pressure adjustment unit penetrating the separation portion and configured to inject gas into the culture space or discharge gas inside the culture space.

14. The cell culture device of claim 13, wherein
at least one of nitrogen, carbon dioxide, and air is introduced into the pressure adjustment unit.

15. The cell culture device of claim 13, further comprising:
a controller configured to receive pressure in the culture space which is measured by the sensing unit and configured to control the pressure adjustment unit,
wherein the controller adjusts the amount of gas introduced from the pressure adjustment unit to maintain the pressure in the culture space at 1 psi to 2 psi.
